(19) United States
(12) Patent Application Publication
(10) Pub. No.: US 2026/0283520 A1
(43) Pub. Date: Sep. 24, 2026

(54) AN IMPROVED GRAPHOLOGY SYSTEM

(71) Applicants: Rohan Rajesh KOTHARI, Ahmedabad (IN); Rajesh KOTHARI, Ahmedabad (IN)

(72) Inventor: Rajesh Kothari, Ahmedabad (IN)

(21) Appl. No.: 19/476,684

(22) PCT Filed: Nov. 6, 2023
(86) PCT No.: PCT/IN2023/051019

§ 371 Date: Oct. 18, 2025

(30) Foreign Application Priority Data
Apr. 21, 2023 (IN) 202321029242

(51) Int. Cl. A61B 5/16; G06V 30/22; G06V 30/32

(52) U.S. Cl. CPC A61B 5/167; G06V 30/22; G06V 30/333

(57) ABSTRACT

The present invention relates to an improved graphology system and is thereby, error free and provides less computation time and less memory consumption for user's handwriting analysis The present invention provides an improved graphology system which comprises: Means of Processing (P), which further comprises of an Input means (1), Means of image inversion (2), Means of generating sub-images (3), Means of OCR (Optical Character Recognition) model (4); Means of analyzing handwriting traits (A) conducted on the basis of various traits required for handwriting analysis; and Means of finalizing output (F) further comprising of Means of compiling, processing and stacking of output (21), and Means of displaying final output (22).

### FIELD OF INVENTION

The present invention relates to field of handwriting analysis. More particularly, it relates to an improved graphology system. The present invention completely eliminates the human intervention and is thereby, error free and provides less processing time and occupies less memory storage for user's handwriting analysis. Moreover, present invention encompasses all the traits for handwriting analysis.

### BACKGROUND OF THE INVENTION

It is believed that the handwriting of every individual is unique and different. Graphology is the analysis of handwriting used for analyzing human characteristics that are revealed in their handwriting. The handwriting is said to reveal emotional as well as psychological state of the individual. In order to identify such emotional and psychological state, various systems exist from centuries. Such systems are used by Individuals and Various Professionals such as Human Resource, Recruitment agency, Education institutes i.e., School, Colleges and Universities, Life partner compatibility, Company management, Motivational Speaker, and medical professionals such as psychologists, psychiatrists and even graphologists to recognize the personality, character and mental status of the individuals. These systems provide signature analysis feature to reveal about user's personality and changes required to portray a dynamic and appealing aura to impress others.

Graphology, being carried out by the professionals is a completely manual task and as a result it is generally a time-consuming process. It requires the expertise, skill, knowledge and logic of a graphologist. Currently, the handwriting expert gets a handwriting sample in a soft copy or hard copy of the original handwritten paper, then the handwriting analyst checks the numerous elements of handwriting including slant, size, pressure, shape, margin (left, right, top and bottom), space between two consecutive letters, words and between two lines, stroke, loop, zones (upper, middle and lower), baseline, stacking of traits and connection between two letters.

Individual letter analysis is an important aspect of graphology as it allows graphologist to examine specific traits and characteristics (features) associated with each letter in an individual's handwriting. These specific traits and characteristics (features) mainly includes margin, pressure, loop, numbers, space, shape and connection between letters.

Similarly, other additional features includes finding the initial stroke as angular, having a small loop, an incomplete loop, a knot in the initial stroke, horizontal with a sharp angle initial stroke, a tiny knot in the initial stroke, a large knot in the initial stroke, a bowl-shaped initial stroke, a hook-shaped initial stroke, loops in the upper and lower zones, incomplete loops in the upper and lower zones, a triangular loop, an extremely long downstroke, a sharp pointed downward stroke, only downward stroke, the final stroke in the left and right sides, long, large, and narrow loops, v-formation, arced, hook, and bowl-shaped final stroke of individual letters, etc. as a subsidiary analysis done by the graphology system.

Herein, the graphology system examines and analyzes the raw handwriting sample of an individual, leading to false positive results and false negative results, wherein false negative results are considered when the predicted value of the sample is positive and the ground truth value of the sample is negative. For false-positive results consideration, the predicted value of the sample is negative and the ground truth value of the sample is positive.

The present invention of an improved graphology system utilizes a deep learning system (OCR) for the extraction of words and characters from a handwriting sample. Here, a separate logic is created for finding the attributes of handwriting analysis, thereby resulting in the minimum chances of obtaining false positive and false negative outputs as a result.

Moreover, graphologists gain insights into the writer's personality, emotional state, cognitive processes, and even potential psychological or behavioral tendencies through this analysis performed by the graphology system.

### Terminologies as Elements of Handwriting Referred in the Present Invention:

Letter: In the science of handwriting, each letter has a distinguished energy and meaning.

Slant: Slant of handwriting means an angle of handwriting with the reference to the baseline and angle of all the alphabets. It represents the emotional responsiveness of a person.

Pressure: The amount of pressure exerted on the pen point shows the depth and intensity of a person's emotions. When a person writes with a fountain pen, the amount of pressure directly affects the width of the stroke as well as the surface taken for keeping the papers for easy observation of the intensity of emotions explored by the said person. But nowadays, only ballpoint is used to verify the pressure while turning the page and indents of the paragraph or points are felt behind it. Therefore, the pressure on the pen is directly proportional to the indentation on the backside of the paper wherein, the measurement and extraction of pressure are performed on the basis of thickness. Higher the thickness is then the pen pressure is also higher and vice versa, and this feature is used for finding the traits.

Shape: It is the overall form or structure of letters, words, and sentences as they are written on paper or any writing surface. Each letter has its own distinctive shape, including the curves, angles, proportions, triangular, multiple “s” formations, spirals, claws, small “c” shapes, angular hooks and bowls, etc., makes it recognizable and from these shapes extraction from an individual letter to find the personality is accomplished.

Margin: Margins grant a frame to written text and limits the graphical conduct of the individual. There are 4 margins comprising of left, right, top and bottom wherein, the left, right, top, and bottom margins of a page are considered and extracted from an image and used for finding the personality.

Numbers: Numbers includes the extraction of numbers from 1 to 9 in handwriting to find the traits.

Space: Space meaning a gap between two things or a distance between two objects. However, as far as handwriting is concerned there are 3 types of spaces including space between two consecutive words, letters or lines wherein, the space between words and the space between letters in words are extracted to find the resulting personality traits.

Stroke: The beginning of any stroke is usually a dot and then it creates motion, direction and pressure. All these are so complex and interlinked with each other.

Loop: In handwriting analysis, a loop refers to a rounded or circular formation that occurs in certain letters or strokes. This feature is used for extracting the height and width of loops to find the traits in a letter that contain a loop in the upper or lower zone.

Zones: The area covered by the letters in handwriting is known as zones. The zones consist of Upper zone, Middle zone and Lower zone.

Baseline: Baseline refers to an imaginary line on which user writes on the paper.

There are two main types of baselines: General baseline and Stroke baseline.

An imaginary line that is drawn beneath the middle zone letters or any word or sentence is called a General Baseline. It is also referred as the Line of Reality. Any typical ruler paper has straight lines which are used to write and the said straight line (in ruled paper) is nearly close to the handwriting baseline but said baseline is not considered.

Whereas, an imaginary line that is drawn from the initial upstroke to the ending downstroke and returns to start a new letter is known as Stroke baseline.

Traits: A trait is a distinguishing characteristic, quality, or feature that is inherent to an individual, object, or concept. Traits refers to various aspects, including physical attributes, personality traits, behavioral patterns, or genetic characteristics.

Stacking of traits: It is more than one trait in handwriting that also defines another trait. A combination of various traits is also called supporting traits and the presence of specific traits in handwriting indicates a specific trait.

The connection between two letters: Refers to how the person connects two consecutive letters to find the traits. Herein, the letters are connected in an upward or downward direction; thereby, output traits from the said letters are generated accordingly.

Grapho-therapy: Grapho-therapy refers a change in a person's handwriting that creates a new neuropathway and eventually, changing person's personality and traits.

Signature: It refers to a unique and distinctive mark, symbol, or stylized writing that serves as a person's personal identifier. It is commonly used to indicate one's consent, approval, or authentication of a document, agreement, or other written material.

Signature is anything that includes our name, family name and middle name. It may be initial letters or maybe full name of first name, family name and/or middle name of the individual.

According to graphology, our subconscious identities are revealed by the pen strokes in our writing, and our personalities are projected by our signatures, which serve as a projection of our public image. Therefore, to excel in our career, our signature reflects our line of work and allows the individual to set their life goals.

Doodles: Doodles refer to spontaneous, often abstract or whimsical drawings that are typically created absentmindedly or during idle moments.

There are endless varieties of handwriting that needs to be analyzed and interpreted. To finish a handwriting analysis, it takes around 2-3 hours to get a detailed personality analysis. This makes the process time-consuming, tedious and possibility of human errors. After the analysis, making a graphotherapy handwriting practice is complex because the said practice is designed in a way that overcomes the negative traits and improves or/adds positive traits based on the current profession and position.

Graphology system is employed in various real-life applications such as:
1. Document analysis—The given documents are compared through examining various characteristics i.e., style, strokes, letter formations, etc. to determine whether the document is written by same individual or not and this analysis is useful in cases or situations where the authorship of a document is in question, such as in fraud investigations, anonymous letters, or disputed contracts;
2. Signature Verification—Involves to compare an individual's known genuine signature with a questioned signature through various factors like the stroke, pen pressure, slant, zones, connecting strokes etc. which ascertains whether they were both written by the same person or not.
3. Logo designing—Creating a logo that reflects the personality and values of a brand or company. Through analyzing the handwriting style of the brand's founders or key stakeholders and type of business, user gain insights into their personalities and characteristics thus, translating the analysis into the design elements of the logo. Choosing the right font, size, color and various other factors are considered for hand writing analysis.
4. Health/Medical condition including Neurological Disorders such as Parkinson's disease, Alzheimer's disease, etc. affecting the body parts coordination thereby impacting an individual's handwriting for which analysis is performed for early detection; Psychological and Emotional Health wherein analyzing the size, slant, pressure, and other handwriting features indicate stress levels, emotional stability, or tendencies toward certain psychological conditions; and Physical health issues like blood pressure, higher tension, and gynecological issues also impact on individual's handwriting.
5. Personality analysis-Graphology system is beneficial to Individuals and Various Professionals such as Human Resource, Recruitment agency, Education institutes i.e., School, Colleges and Universities, Life partner compatibility, Company management, Motivational Speaker, and medical professionals such as psychologists, psychiatrists and even graphologists to recognize the personality, character and mental status of the individuals.
6. Signature analysis-Graphology system helps to reveal about the user's personality and changes required to portray a dynamic and appealing aura to impress others.

Grapho-therapy handwriting exercise helps to change the neuropathway of the person and that eventually improves the personality of the person. This process demands the skill, expertise, knowledge, logic and lot of time of the graphologist/handwriting analyst.

### PRIOR ART AND ITS DISADVANTAGES

There have been various attempts regarding graphology systems that have been developed or proposed by various patentable and non-patentable cited documents, which are shown as follows:

An Indian patent application IN202141019452 discloses a graphotherapy based handwriting analysis system (100) with structural and character features extraction, comprising, a scanning unit (101) for scanning one or more handwriting sample(s), a pre-processing module (102) for enhancing quality of the sample(s), an extraction module (103) that identifies features from the sample(s); and a classification module (104) for analyzing the features to predict out graphology facts in the handwriting sample(s), wherein, the pre-processing module (102) conducts a set of operations on the handwriting sample(s) for enhancing quality of the sample(s), the extraction module (103) identifies structural as well as character features from the handwriting sample(s), the classification module (104) conducting an exhaustive analysis on the features via a neural network protocol to predict out the graphology facts.

Said prior art is not clear about the system used or its accuracy. It fails to provide graphology system that works without any human intervention. Moreover, said prior art is time consuming, inefficient and occupies more memory space.

An Indian patent application no. IN201821029049 discloses a system to analyze suicidal tendency and suicidal notes written or presented using varied mediums. This innovative system is assembled to constantly obtain data from a user's social sites and other sources to analyze the mental state and emotional stability of an individual to evaluate their suicidal tendency. Said system is also capable to be commanded from distant computing devices to obtain an analysis of a suicide note which is handwritten or provided in any digital form to evaluate the reason behind the act and to identify the authenticity of the cause of death without the help of human assistance, hence making the system time efficient and responsive to use.

Said prior art is only limited to suicidal tendency analysis and highlights only specific properties of personality traits in suicidal notes. Furthermore, said prior art fails to disclose about the structure of the computer server with its interface controller having specific personality traits which is limited to suicidal tendency analysis. Moreover, said prior art is time consuming, inefficient and occupies more memory space.

Another such patent application US2013071030A1 discloses a method for graphology-based assessment of a subject's personality traits is disclosed which involves graphological analysis of inferred handwriting features that are derived from the subject's presentation features. The method includes: obtaining presentation features of a subject; deriving inferred handwriting features of the subject from the presentation features according to pre-determined rules of correlation between the presentation features and the inferred handwriting features; and then performing graphological analysis on the inferred handwriting features so as to provide an assessment of the subject's personality traits. Presentation features includes a subject's general appearance, attire, body language, facial expression, and/or conversational style, for example. Handwriting features includes zonal sizes, connective forms, the handwriting's pressure, spacing, size, and slant, level of script, primary and secondary variables, and/or overall form level data.

Said prior art considers various variables such as appearance, body language and handwriting of the presenter. Furthermore, the accuracy of the prior art is not clear hence, it is time consuming and inefficient.

A U.S. Patent application no. US2012/0114246A1 proposes a method for enhanced graphological personality assessment through analysis of handwriting by categorizing the handwriting features in primary and secondary variables, dominant and non-dominant personality traits. It also provides a way to take into account the relative influence of personality traits indicated by different handwriting features, depending upon the type of handwriting features, and how strongly those features are expressed. The method improves the assessment of personality through handwriting analysis, by separating handwriting features into categories that highlight which aspects of the subject's personality are inherent and deeply-rooted versus changeable and superficial; as well as which aspects of the subject's personality are habitually expressed, versus non-habitually expressed. Handwriting features are separated into primary variables that represent more inherent aspects of the subject's personality, and secondary variables that represent less inherent aspects of the subject's personality. Handwriting features are also separated into dominant variables that represent habitually expressed aspects of the subject's personality, and non-dominant variables that represent non-habitually expressed aspects of the subject's personality.

Said prior art is based on human intervention process. Hence, it requires person's skill set and expertise in the field of graphological personality assessment to perform said method and due to involvement of a person to operate/perform the method leads to inaccurate, questionable efficiency and time-consuming process. Further, said prior art fails to provide any details of the report of the person which clearly proves that the accuracy of the system is questionable.

A U.S. Patent application no. US20110217679A1 relates to a diagnosis system and method comprising a handwriting data collection and analysis software and accompanying tools for diagnosing a given condition such as an illness, a mental illness, a physiological condition, a mental or emotional state, medication effect, a personality trait, a skill, an expertise, deception or truth writing. The handwriting of a control group known to have a condition is analyzed in order to determine the detectable characteristic indicators of that group. Such measured indicators comprise spatial, temporal and pressure measures for each writing stroke. The handwriting of a subject is then analyzed to determine if the measured indicators indicate statistically that the subject is diagnosed with a condition.

Said prior art is only limited to mental illness and highlights only specific properties of personality traits in handwriting data collection. Further, the accuracy of the previous system of prior art is unclear and vague. Hence, said prior art is time consuming, inefficient and occupies more memory space.

The tool named Graphonomizer was invented in year 1990s. The issue with the tool is it gives only very limited traits that is analyzed and very limited elements of handwriting is covered. There is a possibility of error because it does not analyze the handwriting itself, there is no provision to upload any file but, a graphologist has to feed information using his own knowledge.

Said Graphonomizer, only provides limited traits which is analyzed. Another drawback of the said prior art is that it fails to provide accurate analysis leading to inefficient results and more time consumption.

### DISADVANTAGES OF THE PRIOR ART

Existing systems used for the analysis of handwriting suffers from all or at least any of the below mentioned disadvantages:
Many of the prior arts fail to provide a graphology system that provides accurate results since they do not include all the traits required for the handwriting analysis.
Many of the prior arts fail to provide a graphology system that is fast since prior arts consume more processing time for analysis.
Most of the prior arts fail to provide a graphology system that occupies less memory storage.
Most of the prior arts fail to provide a graphology system that provides higher efficiency of the system.
Most of the prior arts fail to provide a graphology system that requires expertise and human intervention by graphologist.
Most of the prior arts fail to provide a graphology system that does not depend on certain characters, letters, or elements for finding the personality trait.
Most of the prior arts fail to provide a graphology system that provides stacking and compilation of seen and unseen traits.
Many of the prior arts fail to provide a graphology system that is used by laymen and experts and is thus, user-friendly.

Thus, there is an unmet need to develop an invention that suffices the purpose of providing an improved graphology system, used for the analysis of handwriting.

### OBJECTS OF THE INVENTION

The main object of the present invention is to provide an improved graphology system.

Yet another object of the present invention is to provide an improved graphology system that provides accurate results for all the types of traits.

Yet another object of the present invention is to provide an improved graphology system that is time efficient.

Yet another object of the present invention is to provide an improved graphology system that occupies less memory space.

Yet another object of the present invention is to provide an improved graphology system that provides higher efficiency of the system.

Yet another object of the present invention is to provide an improved graphology system that results efficiently without depending only upon certain characters, letters, or elements for finding the personality trait.

Yet another object of the present invention is to provide an improved graphology system that provides stacking and compilation of seen and unseen traits which leads to false positive and false negative results.

Yet another object of the present invention is to provide an improved graphology system that works without any expertise and human intervention to provide the results by graphologist.

Yet another object of the present invention is to provide an improved graphology system that is significantly user-friendly.

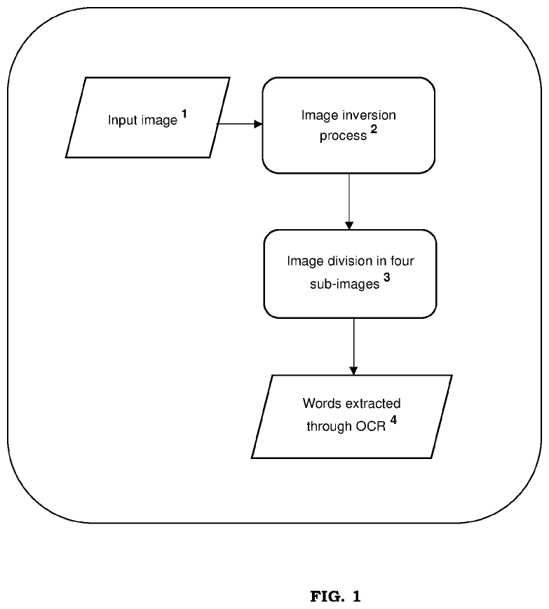

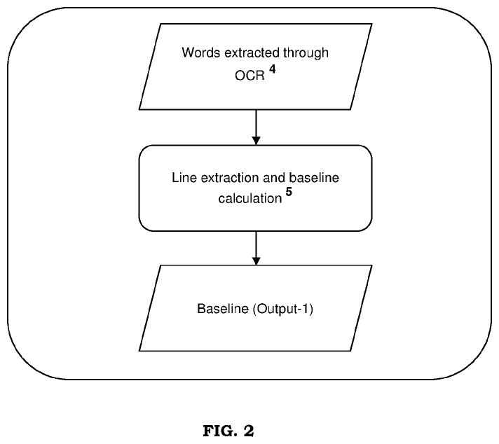

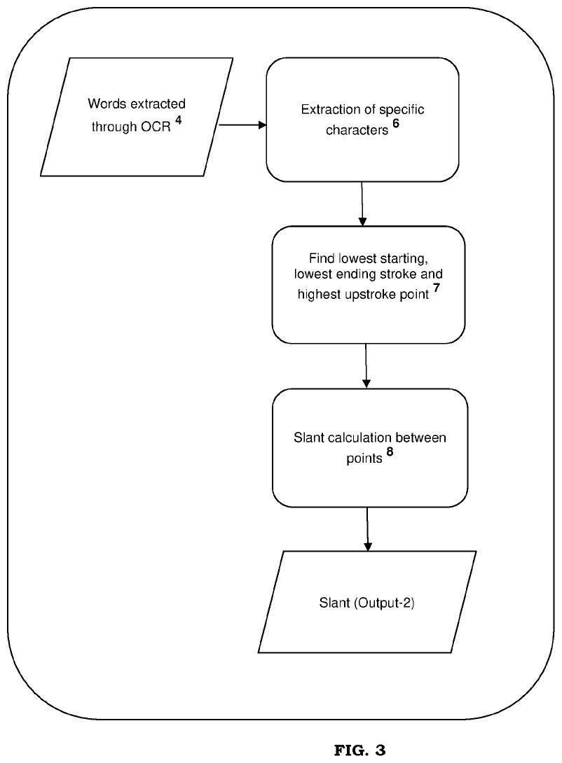

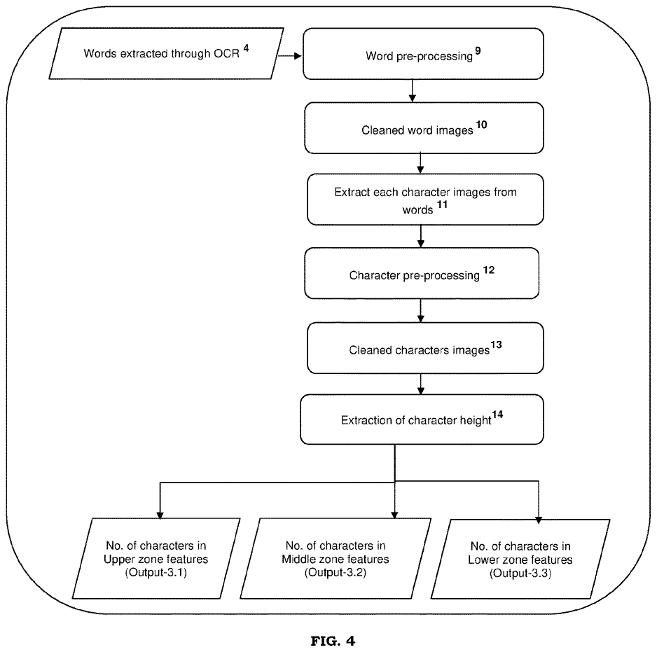

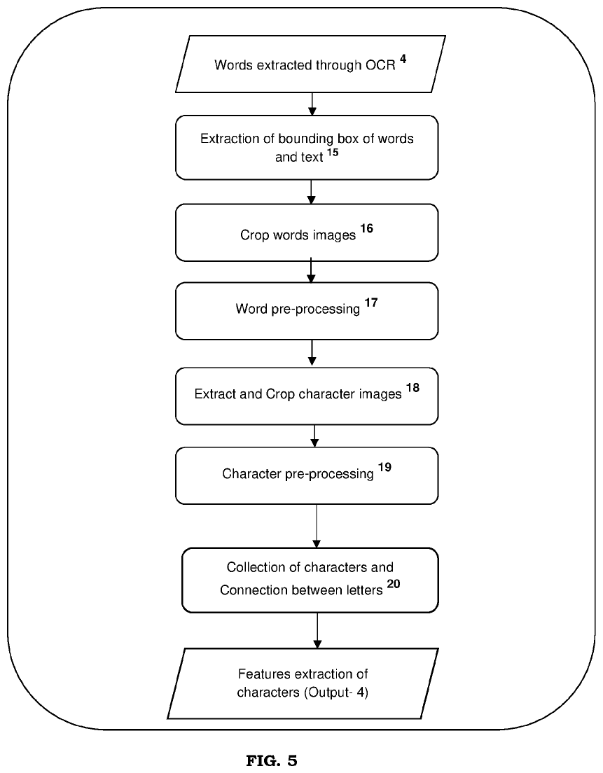

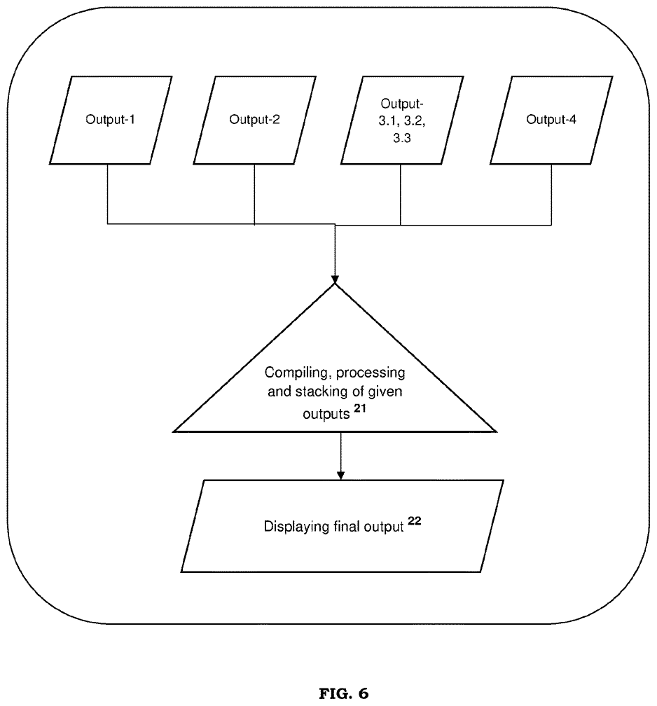

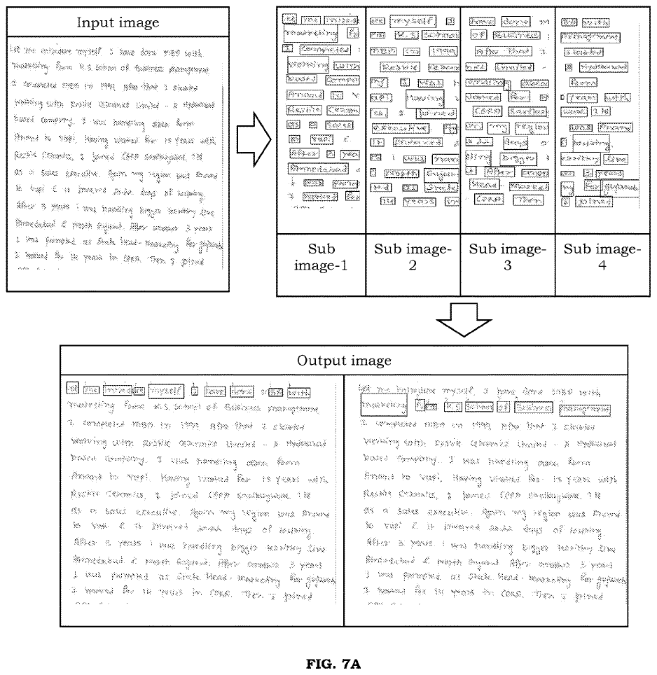

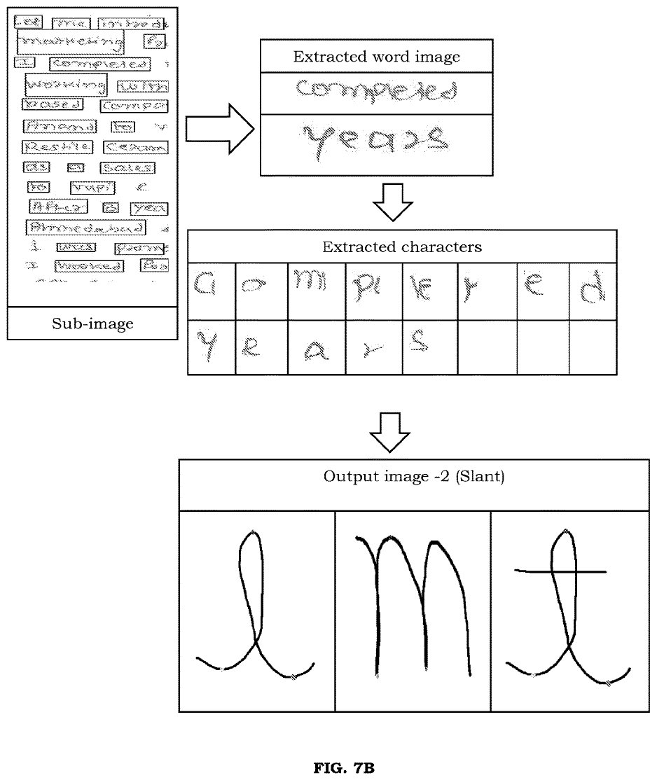

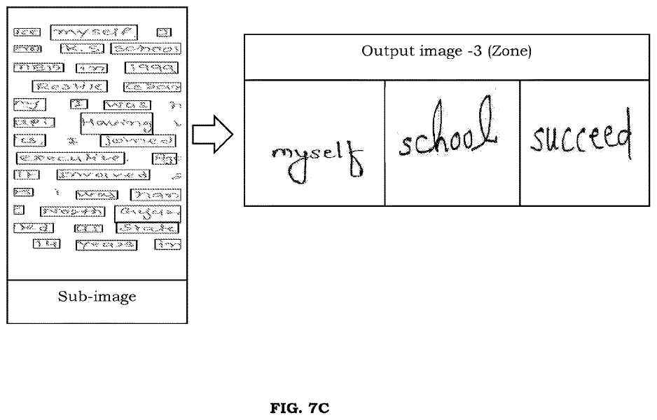

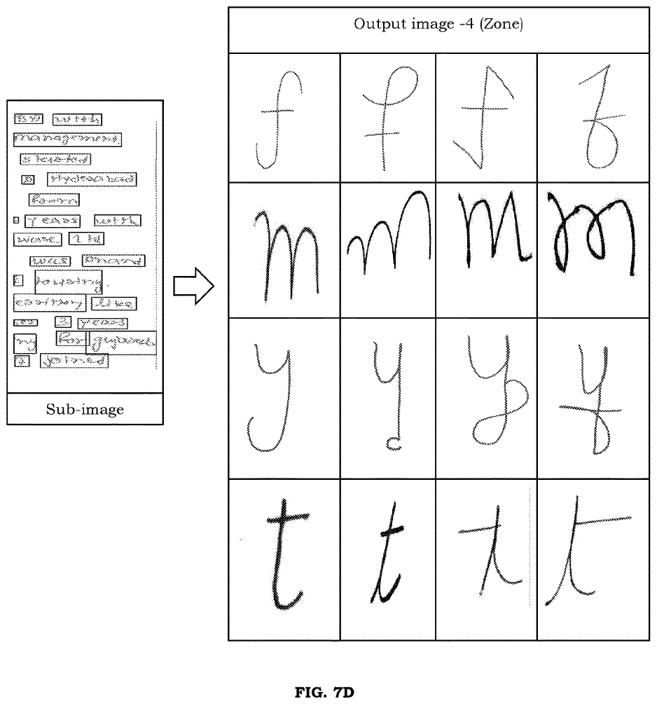

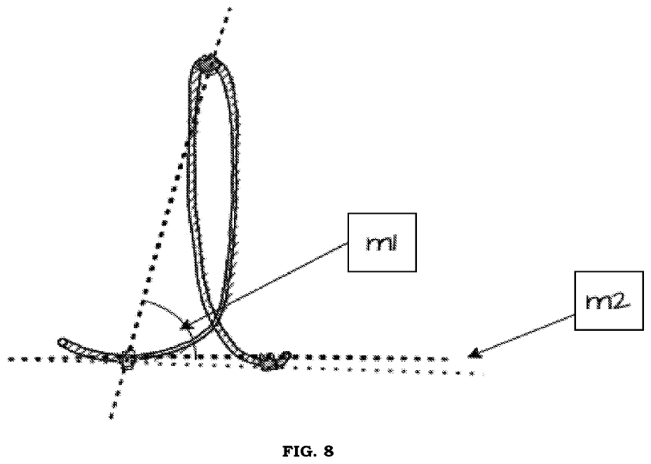

### BRIEF DESCRIPTION OF DRAWINGS

| | |
|---|---|
| FIG. 1 | Shows the block diagram depicting the image processing |
| | of an improved graphology system. |
| FIG. 2 | Shows the block diagram analyzing the features of |
| | baseline. |
| FIG. 3 | Shows the block diagram analyzing the features of slant. |
| FIG. 4 | Shows the block diagram analyzing the features of zone. |
| FIG. 5 | Shows the block diagram analyzing the features of |
| | individual letter with connection between letters. |
| FIG. 6 | Shows the block diagram depicting the output finalizing |
| | of an improved graphology system. |
| FIG. 7A | Diagram representing sample outputs for handwriting |
| | analysis on the basis of baseline. |
| FIG. 7B | Diagram representing sample outputs for handwriting |
| | analysis on the basis of slant angle. |
| FIG. 7C | Diagram representing sample outputs for handwriting |
| | analysis on the basis of zone. |
| FIG. 7D | Diagram representing sample outputs for handwriting |
| | analysis on the basis of individual letters. |
| FIG. 8 | Diagram representing a character sample “1” for slant |
| | angle analysis. |

Meaning of Reference Numeral of the Said Component Parts of the Present Invention to Provide an Improved Graphology System which is Used for Handwriting Analysis:

| P | Means of Processing |
|---|---|
| A | Means of Analyzing Handwriting Traits |
| F | Means of Finalizing Output |
| 1 | Input Means |
| 2 | Means of Image Inversion |
| 3 | Means of Generating Sub-Images |
| 4 | Means of OCR Model |
| 5 | Means of Line Extraction and Baseline Calculation |
| 6 | Means of Character Extraction |
| 7 | Finding handwriting strokes |
| 8 | Means of Slant Calculation |
| 9 | Means of Word Pre-Processing |
| 10 | Means of Clean Word Images |
| 11 | Means of Extraction and Cropping Characters |
| 12 | Means of Character Pre-Processing |
| 13 | Means of Clean Character Images |
| 14 | Means of Extraction of Character Height |
| 15 | Means of Bounding box of words and text extraction |
| 16 | Means of Cropping |
| 17 | Means of Word Pre-processing |
| 18 | Means of Extraction and Cropping Characters |
| 19 | Means of Character Pre-processing |
| 20 | Means of Character Extraction |
| 21 | Means of Compiling and Processing and Stacking Output |
| 22 | Means of Displaying Final Output |

### SUMMARY OF INVENTION

The present invention provides an improved graphology system for accurate handwriting analysis without any human intervention. Said invention is divided into 3 parts: Means of Processing (P), Means of Analyzing Handwriting Traits (A), and Means of Finalizing Output (F). Herein, Means of Processing (P) comprises of an Input means (1); Means of image inversion (2); Means of generating sub-images (3); Means of OCR (Optical Character Recognition) model (4).

Further, Means of Analyzing Handwriting Traits (A) are conducted on the basis of traits required for handwriting analysis and different outputs are obtained from these traits which are subsequently sent to Means of Finalizing Output (F) which comprises of Means of compiling and processing and stacking output (21) and Means of displaying final output (22). Said invention consumes less processing time in analysis and occupies less memory storage. Furthermore, said invention works without any human intervention to provide accurate results. Moreover, said invention provides stacking and compiling of seen and unseen traits, leading to false positive and false negative results. Thus, said invention is accurate, fast, time-saving, occupies less memory storage and user-friendly.

### DESCRIPTION OF INVENTION

The present invention provides an improved graphology system that works without any human intervention. Said invention consumes less processing time in handwriting analysis and occupies less memory storage through Multithreading technique. Here, Multithreading is a technique that allows multiple tasks to be executed concurrently within a single program and it is performed by dividing the program into multiple threads, each executing its own code independently. Furthermore, said invention facilitates less processing time, thus making the system time-saving and concurrently reduces the complexity of processing time. Each process for detecting or processing various handwriting traits such as baseline, zone, slant, and extraction of features of individual characters are generated, and all the processes for the same are concurrent. Moreover, said invention works without any expertise or human intervention to provide an accurate result. Therefore, any person with basic knowledge on computer or smartphone uses the system and performs the handwriting analysis. Thus, said invention is accurate, fast, time-saving, occupies less memory storage and user-friendly which provides a technically advanced and significantly efficient system.

The present invention relates to an improved graphology system detecting different handwriting traits based on user's handwriting analysis. Said system comprises of:
Means of Processing (P)
Means of Analyzing Handwriting Traits (A)
Means of Finalizing Output (F)

Wherein;

Said Means of Processing (P) further comprises of:
An Input means (1);
Means of Image Inversion (2);
Means of Generating Sub-images (3);
Means of OCR model (4);

Wherein;

Said Input Means (1) facilitates the scanning or capturing of the handwritten file or scanned copy through digital device such as but not limited to mobile or laptop or desktop. Herein, an image is captured as a photo of a handwritten file or note, which is read and then forwarded to the Means of image inversion (2).

Said Means of Image Inversion (2) performs an image inversion process in which images are converted from a dark area to a light area and vice versa leading to enhance the visual quality of the image. These images are then forwarded to the Means of Generating Sub-images (3).

Said Means of Generating Sub-images (3) produces four sub images to extract the lines for different forms of traits which are extracted from these images. It identifies all types of handwriting such as wavy, concave, convex, ascending, false ascending, descending, typical quitter etc. Therefore, these images are processed concurrently, and thereby it reduces the overall time duration, and the multithreading technique is employed to ensure less memory storage.

Said Means of OCR (Optical Character Recognition) Model (4) processes these sub images and extract an easily accessible electronic text version from said sub-images.

The text extracted from these sub images through said Means of OCR (Optical Character Recognition) Model (4) are forwarded to Means of analyzing handwriting traits (A).

Herein, said means of analyzing handwriting traits (A) processes all the traits of handwriting analysis that varies within an individual's handwriting. These traits include:
Baseline
Zone
Slant (Angle of Characters)
Features of individual letter
   Margin
   Space
   Loop
   Numbers
   Shape
   Pressure
   Connection between two letters

The block diagram of FIG. 2 depicts the baseline calculation from the words extracted through said Means of OCR (Optical Character Recognition) Model (4) wherein, baseline trait refers to an imaginary line on which user writes on the paper. These words are then forwarded to Means of Line Extraction and Baseline Calculation (5).

Said Means of Line Extraction and Baseline Calculation (5), examines all the words of the OCR and identifies each line of the words, thereby determining all the lines for calculating the baseline trait through:

$$math . degrees ( math . atan ( \frac{{m}_{2} - {m}_{1}}{1 + ( {m}_{1} * {m}_{2} )} ) )$$

Wherein,
“math” is a Python module used for mathematical tasks; “math.atan (x)” returns the arc tangent of x and returns the angle in radians;
“math.degrees” returns the angle in degree format, as it basically shows the conversion of an angle from radian to degree.

Herein, baseline is identified on the basis of the angle calculated by comparing the output obtained by the above formula with the pre-set value of the angle, thereby determining the baseline of each line that falls under different types of baseline traits. Output 1 is generated through Means of Line Extraction and Baseline Calculation (5), which examines the highest number of similar types of lines and combines the final number of lines which are extracted and analyzed from individual sub-images containing multiple output traits.

The block diagram of FIG. 3 depicts the slant angle calculation of individual characters from the words extracted through said Means of OCR (Optical Character Recognition) Model (4) wherein slant or angle of character refers the angle of handwriting.

These words are then sent for character extraction from individual words through Means of Characters extraction (6). Here, character extraction is used for extracting specific characters such as “k, t, m, n, h, 1, t and d” and these extracted characters are calculated to find their lowest starting point, lowest ending point and highest upstroke point.

Here, the slope is calculated for slant calculation, between “lowest starting point” and “highest upstroke point” using formula:

$$\frac{{y}_{2} - {y}_{1}}{{x}_{2} - {x}_{1}}$$

and it is represented as ‘m1’. The same representation has been done for ‘m2’, which is found between “lowest starting point” and “lowest ending stroke”.

For “lowest starting point”, a list of individual rows containing coordinates of white pixels are created and a comparison point is selected from the last row. From the last row, ‘x max’ and ‘x min’ points are marked in a particular row. Here, if ‘current x min’ (x min1) is less than ‘earlier x min’ (x min2), then ‘current x min’ (x min1) becomes a comparison point, whereas if ‘current x min’ (x min1) is greater than ‘earlier x min’ (x min2), then current x min′ (x min1) becomes the “lowest starting point” point.

For “lowest ending stroke” point, a list of each column containing coordinates of white pixels are created and a comparison point is selected from last column. From the last column, ‘y min’ and ‘y max’ points are marked in particular column. Here, if ‘current y min’ (y min1) is larger than ‘earlier y min’ (y min2) then ‘current y min’ (x min1) becomes a comparison point and if ‘current y min’ (y min 1) is lower than ‘previous y min’ (y min2) then ‘current y min’ (x min1) becomes “lowest ending stroke” point.

For letter “m and k”, this step continues for two times and second point becomes “lowest ending stroke” point.

While a list containing coordinates of white pixels is generated and sorted by y value, the point containing the lowest y value becomes the “highest upstroke point”.

Through the Means of Slant calculation (8), an angle is calculated from these extracted character points using the below mentioned formula:

$$180 - math . degrees ( math . atan ( \frac{{m}_{2} - {m}_{1}}{1 + ( {m}_{1} * {m}_{2} )} ) )$$

and the said angle is selected from the slant category table for obtaining a specific slant trait containing maximum multiple traits of multiple letters (t,m,h,r,n,l,d,b) which are analyzed and compiled as Output-2. These traits are then verified through the Slant category table:
FA slant at 60-90°
AB slant at 90-110°
BC slant at 110-123°
CD slant at 123-133°
DE slant at 133-145°
E+ slant at) (>145°)
F− slant at (<60°)

The block diagram of FIG. 4 depicts the zone calculation of individual words from the words extracted through Means of OCR (Optical Character Recognition) Model (4) wherein zone refers to specific areas within a person's handwriting where certain letters, strokes or other graphical elements are found and each zone is associated with particular traits and characteristics.

These extracted words are then forwarded to Means of Word Pre-processing (9) for the removal of any extraneous white portions from the word images, which is carried out by the inversion process. These words are cleaned by cancelling the noise through Means of Clean Word Images (10). These words are sent to Means of Extraction and Cropping characters (11) for extracting each character from word and these extracted characters are then forwarded to Means of Character Pre-processing (12) for removal of any extra white portions from the character using the inversion process. These characters are then cleaned by canceling the noise using Means of Clean Character Images (13) and cleaned character images extracts the height and width of the character through Means of Extraction of character height (14) and obtains various types of zones containing multiple average and maximum traits which are analyzed and compiled as Output 3.1, 3.2 and 3.3.

The block diagram in FIG. 5 depicts the feature of individual letters calculation from the words extracted through Means of OCR (Optical Character Recognition) Model (4).

All the characters are analyzed in both upper and lower case to determine the resulting traits as well as different types of features, such as loop height and width, thickness, letter shape, etc. For example, characters such as m, t, f, and y have been extracted and analyzed on the basis of their features such as margin, pressure, loop, number, space, shape, etc.

Means of Bounding box of words and text extraction (15) wherein, Means of OCR Model (4) extract the bounding box coordinates of the extracted text. These extracted words are cropped through Means of Cropping (16) and then sent to Means of Words Pre-processing (17) for elimination of any extraneous white portions from the word to obtain clean images. These clean images are further sent to Means of Extraction and Cropping characters (18) for extracting and cropping the individual character from the clean words. Extracted characters are forwarded to Means of Character pre-processing (19) for removal of any extra white portion from the extracted character to obtain clean character images. These clean characters are collected and a separate list is created for each character type through Means of Character collection (20). Here connection between two characters are extracted and traits are found accordingly. These unique traits of each character as well as the connection between two characters, are listed and sent as Output 4.

Different trait outputs (Output-1, Output-2, Output-3, and Output-4) from said Means of Analyzing Handwriting Traits (A) are forwarded to said Means of Finalizing output (F), it further comprises of:
Means of Compiling, Processing and Stacking Output (21)
Means of Displaying Final Output (22)

Means of Compiling, Processing and Stacking Output (21) gathers different trait outputs (Output-1, Output-2, Output-3, and Output-4) for compiling, processing and stacking the received outputs for final output generation.

Herein, Output 1 (Baseline) contains multiple traits based on the final number of extracted and analyzed lines; Output 2 (Slant) contains multiple traits based on the angle of a specific letter, wherein a maximum count is taken for output generation; Output 3 (Zone) contains multiple traits based on the zone of a letter; and Output 4 (Individual Letter) contains multiple traits based on the extracted features of an individual letter and contains multiple elements like space, margin, pressure, loop, numbers, connection between two letters etc. The final output is generated by combination of maximum or equal traits of each output from different handwriting traits, and all the outputs (Output 1, Output 2, Output 3, and Output 4) are merged as a report, which includes trait count, trait type and trait description.

Seen and unseen traits are combined (stacked) and analyzed with the list of different output traits obtained by means of analyzing handwriting traits (A), and then compiled and processed with the identified traits of the outputs through permutation and combination, leading to the generation of the minimum chances of false positive and false negative results.

The final output as report containing all the merged (compiled) data is then displayed on digital devices such as but not limited to mobile device or laptop or desktop through Means of Displaying Final Output (22). For future reference, separate PDF file containing the final merged report is generated and saved in storage device.

The present invention of an improved graphology system takes multiple factors into account for the analysis of handwriting such as combinations of multiple parameters of the handwriting such as baseline, zone, slant, pressure, margin, loop, shape, stroke, connection of letters, features of individual character, space, numbers and stacking of traits etc. Thus, the present invention provides accurate, fast, secure, and reliable results, at anytime and anywhere within a few minutes.

The foregoing disclosure and description of the invention is illustrative and explanatory thereof. Various changes in the details of the illustrated diagrams can be made within the scope of the appended claims without departing from the true spirit of the invention. The present invention should only be limited by the following claims and their legal equivalents.

### Sample:

FIG. 7A-7D illustrates on diagrammatic representation of various types of handwriting samples with an analysis of traits available in the present invention of an improved graphology system. As demonstrated in the FIG. 7A-7D, all the images from output 1,2,3 and 4 represents sample analysis of different trait output.

FIG. 8 depicts a diagrammatic representation of a sample character “1” for slant angle analysis carried out by said system. Herein, “m1” represents slant angle calculation of slope between “lowest starting point” and “highest upstroke point” whereas “m2” represents slant angle calculation of slope between “lowest starting point” and “lowest ending stroke”.

Herein, said invention analyzes different aspects of handwriting traits to comprehend an individual's personality, as handwriting reveals everything about an individual for understanding their potential and strengths. Moreover, said invention utilizes the Multithreading technique, resulting in less processing time and less consumption of memory storage for obtaining an accurate result.

### ADVANTAGES OF THE PRESENT INVENTION

The present invention provides an improved graphology system that provides accurate results for all the traits.

The present invention provides an improved graphology system that occupies less memory storage.

The present invention provides an improved graphology system that reduces the processing time and is thus, time-saving and fast.

The present invention provides an improved graphology system that ensures the privacy of the data of the users.

The present invention provides an improved graphology system that does not need any expertise to implement the results and is thus, user-friendly.

The present invention provides an improved graphology system that does not depend on certain characters, letters, or elements for finding the personality trait.

The present invention provides an improved graphology system that provides higher efficiency of the system.

The present invention provides an improved graphology system that provides stacking and compilation of unseen traits which leads to the generation of false positive and false negative results.

The present invention provides an improved graphology system that provides a graphotherapy practice after the analysis is done, which helps to change the neuropathway of the person that enhances the personality and thought process.

We claim:

1. An improved graphology system, wherein said system is time-saving and occupies less memory space, detects different handwriting traits based on the user's handwriting and comprises of:
Means of Processing (P);
Means of Analyzing Handwriting Traits (A);
Means of Finalizing Output (F);
Wherein,
Said Means of Processing (P) further comprises of:
An Input Means (1);
Means of Image Inversion (2);
Means of Generating Sub-images (3);
Means of OCR Model (4);
Wherein;
Said Input Means (1) configured to scan or capture a handwritten file or scanned copy as an image through digital devices, which is read and forwarded to said Means of Image inversion (2) for performing an image inversion process, where these images are converted from a dark area to a light area and vice versa, said images are then sent to said Means of Generating Sub-images (3) which produces four equal sub images for extracting the lines from text images for different forms of trait extraction, these sub-images are sent to said Means of OCR (Optical Character Recognition) Model (4) configured to process and extract these sub images into an accessible electronic text version which are then sent through said Means of Analyzing Handwriting Traits (A).
Wherein,
Said Means of Analyzing Handwriting Traits (A) configured to process all the traits of handwriting analysis that varies within an individual's handwriting. These traits include:
The baseline calculation which is examined from the words extracted through said Means of OCR (Optical Character Recognition) Model (4). These words are then forwarded to Means of Line Extraction and Baseline Calculation (5) which examines all the words of the OCR and identifies each line of the words, thereby determining all the lines for calculating the baseline trait and generates Output 1.
The slant angle calculation of individual characters which is examined from the words extracted through said Means of OCR (Optical Character Recognition) Model (4). These words are forwarded for character extraction from individual words through Means of Characters extraction (6) and these extracted characters are calculated to find their lowest starting point, lowest ending point and highest upstroke point and from these extracted character points, an angle is calculated through the Means of Slant calculation (8) and the said angle is selected from the slant category which is further analyzed and compiled as Output-2.
The zone calculation of individual words which is examined from the words extracted through said Means of OCR (Optical Character Recognition) Model (4). These extracted words are then forwarded to Means of Word Pre-processing (9) for the removal of any extraneous white portions from the word images, which is carried out by the inversion process and these words are cleaned by cancelling the noise through Means of Clean Word Images (10) and sent to Means of Extraction and Cropping characters (11) for extracting each character from clean word and these extracted characters are then forwarded to Means of Character Pre-processing (12) for removal of any extra white portions from the characters and these characters are cleaned by canceling the noise using Means of Clean Character Images (13) and cleaned character images extracts the height and width of the character through Means of Extraction of character height (14) for obtaining plurality of zones containing multiple average and maximum traits which are analyzed and complied as Output 3.1, 3.2 and 3.3.
The feature of individual letters calculation of individual characters which is examined from the words extracted through said Means of OCR (Optical Character Recognition) Model (4) and extracts the bounding box coordinates of the extracted text through Means of Bounding box of words and text extraction (15) which are cropped through Means of Cropping (16) and then sent to Means of Words Pre-processing (17) for elimination of any extraneous white portions from the word to obtain clean images which are forwarded to Means of Extraction and Cropping characters (18) for extracting and cropping the individual character from the clean words and forwarded to Means of Character pre-processing (19) for removal of any extra white portion from the extracted character obtaining clean character images, said images are collected and created a separate list for each character type through Means of Character collection (20), the connection between two characters are extracted and traits are found accordingly, said unique traits of each character and the connection between two characters, are listed and sent as Output 4.
Different trait outputs (Output-1, Output-2, Output-3, and Output-4) from said Means of Analyzing Handwriting Traits (A) are forwarded to said Means of Finalizing output (F) and it comprises of:
Means of Compiling, Processing and Stacking Output (21) and;
Means of Displaying Final Output (22);
Wherein;
Means of Compiling, Processing and Stacking Output (21) assemble different trait outputs (Output-1, Output-2, Output-3, and Output-4) for compiling, processing and stacking the received outputs for generation of final output as report containing all the compiled data and displayed on digital devices through Means of Displaying Final Output (22).

2. An improved graphology system as claimed in claim 1, wherein the digital device may be selected from the group of but not limited to mobile or laptop or desktop

3. An improved graphology system as claimed in claim 1, wherein said Means of generating sub-images (3) generates sub-images processing concurrently to reduce the overall time duration and multithreading technique is employed to ensure less memory storage.

4. An improved graphology system as claimed in claim 1, wherein said Output 1 contains multiple traits based on the final number of extracted and analyzed lines.

5. An improved graphology system as claimed in claim 1, wherein said Output 2 contains multiple traits based on the angle of a specific letter; wherein a maximum count is taken for output generation.

6. An improved graphology system as claimed in claim 1, wherein said Output 3 contains multiple traits based on the zone of a letter.

7. An improved graphology system as claimed in claim 1, wherein said Output 4 contains multiple traits based on the extracted features of an individual letter and contains multiple elements like space, margin, pressure, loop, numbers, connection between two letters.
